(19) United States
(12) Patent Application Publication
(10) Pub. No.: US 2008/0312200 A1
(43) Pub. Date: Dec. 18, 2008

(54) Medicament Comprising A Hormone Combination

(75) Inventors: Georg Schramm, Stolberg (DE); Christa Kneip, Aachen (DE)

Correspondence Address: NORRIS, MCLAUGHLIN & MARCUS, PA, 875 THIRD AVENUE, 18TH FLOOR, NEW YORK, NY, 10022, US

(73) Assignee: Grunenthal GmbH, Aachen (DE)

(21) Appl. No.: 12/169,357

(22) Filed: Jul. 8, 2008

Related U.S. Application Data
(63) Continuation of application No. PCT/EP2007/000552, filed on Jan. 23, 2007.

(30) Foreign Application Priority Data
Jan. 24, 2006 (DE) 10 2006 003 508.9

(51) Int. Cl. A61K 31/565

(52) U.S. Cl. 514/170; 514/171

(57) ABSTRACT

The present invention relates to the use of a hormone combination of ethinyl oestradiol or oestradiol as oestrogen component and at least one metabolite of chlormadinone acetate selected from the group consisting of 3α-hydroxy-6-chloro-17α-acetoxy-4,6-pregnadien-20-one (3α-hydroxychlormadinone acetate), 3β-hydroxy-6-chloro-17α-acetoxy-4,6-pregnadien-20-one (3β-hydroxychlormadinone acetate), 3α-hydroxy-17α-acetoxy-5β-pregnan-20-one and 3β-hydroxy-17α-acetoxy-5β-pregnan-20-one, optionally mixed with chlormadinone acetate as gestagen component for producing a medicament at least for alleviating menstrual cycle-dependent mood swings and optionally for hormonal contraception in women.

The present invention relates to the use of a hormone combination of at least one oestrogen selected from the group consisting of ethinyl oestradiol (I) and oestradiol (II) as oestrogen component and at least one metabolite of chlormadinone acetate selected from the group consisting of 3α-hydroxy-6-chloro-17α-acetoxy-4,6-pregnadien-20-one (3α-hydroxychlormadinone acetate), 3β-hydroxy-6-chloro-17α-acetoxy-4,6-pregnadien-20-one (3β-hydroxychlormadinone acetate), 3α-hydroxy-17α-acetoxy-5β-pregnan-20-one and 3β-hydroxy-17α-acetoxy-5β-pregnan-20-one, optionally mixed with chlormadinone acetate as gestagen component for producing a medicament at least for alleviating menstrual cycle-dependent mood swings and optionally for hormonal contraception in women.

Many women suffer from mood swings during their menstrual cycle, which may also occur even when the woman is facing no external, discernible influences, complaints and/or disorders and/or no complaints and/or disorders associated with the menstrual cycle. These mood swings may be manifested not only in increased irritability at certain phases of the menstrual cycle, but also in a feeling of lowness and/or increased sensitivity going as far as sorrow without there being any discernible explanation for it.

It goes without saying that such mood swings, which many women find to be an impairment of their mental state, impair the quality of life of women. Furthermore, these mood swings constantly recur on a regular basis over the course of the menstrual cycle, such that many women feel a need not to have constantly to be re-exposed to such mood swings or at least to alleviate these mood swings over the course of a menstrual cycle and/or even to be able to improve their overall mood.

There is therefore a need to provide a medicament for women which is suitable at least for alleviating, preferably for preventing, menstrual cycle-dependent mood swings, which conventionally occur without any discernible (further) reason, going as far as achieving an overall uplifting of mood throughout the menstrual cycle.

This object is achieved by the use of a hormone combination of at least one oestrogen selected from the group consisting of ethinyl oestradiol (I) and oestradiol (II) as oestrogen component and at least one metabolite of chlormadinone acetate selected from the group consisting of 3α-hydroxy-6-chloro-17α-acetoxy-4,6-pregnadien-20-one (3α-hydroxychlormadinone acetate), 3β-hydroxy-6-chloro-17α-acetoxy-4,6-pregnadien-20-one (3β-hydroxychlormadinone acetate), 3β-hydroxy-17α-acetoxy-5β-pregnan-20-one and 3β-hydroxy-17α-acetoxy-5β-pregnan-20-one, optionally mixed with chlormadinone acetate as gestagen component for producing a medicament at least for alleviating menstrual cycle-dependent mood swings, preferably going as far as uplifting mood, and optionally for hormonal contraception in women.

The medicament is preferably provided in the form of daily units. A daily unit is preferably produced using a hormone combination preferably consisting of 0.001 to 50 μg, particularly preferably of 5 to 50 μg, very particularly preferably of 5 to 30 μg, of the oestrogen component (I) or preferably of 0.5 to 4 mg, very particularly preferably of 0.5 to 2 mg, very particularly preferably of 1 mg of the oestrogen component (II) and preferably of 1 to 10 mg, particularly preferably of 1 to 5 mg, very particularly preferably of ≧2 mg of the gestagen component and optionally conventional auxiliary substances.

According to the invention, one of the following components a) to k) may be used as the gestagen component,
a) 3α-hydroxychlormadinone acetate or
b) 3β-hydroxychlormadinone acetate or
c) a mixture of a) and b) in any desired mixing ratio or
d) 3α-hydroxy-17α-acetoxy-5β-pregnan-20-one or
e) 3β-hydroxy-17α-acetoxy-5β-pregnan-20-one or
f) a mixture of d) and e) in any desired mixing ratio or
g) a mixture of a) and/or b) with d) and/or e) in any desired mixing ratio or
h) a mixture of chlormadinone acetate (CMA) with a) and/or b) in a mixing ratio of 10 to 90 wt. % of CMA and 90 to 10 wt. % of a) and/or b), relative to the total mixture, or
i) a mixture of chlormadinone acetate with d) and/or e) in a mixing ratio of 10 to 90 wt. % of chlormadinone acetate and 90 to 10 wt. % of d) and/or e), relative to the total mixture, or
j) a mixture of chlormadinone acetate with c) and f) in a mixing ratio of 10 to 90 wt. % of chlormadinone acetate and 90 to 10 wt. % of c) and f), relative to the total mixture, or
k) a mixture of chlormadinone acetate with g) in a mixing ratio of 10 to 90 wt. % of CMA and 90 to 10 wt. % of g), relative to the total mixture.

If adequate contraceptive protection is also to be achieved in women, a daily unit should be produced using a hormone combination consisting of in each case at least 15 μg, preferably 20 μg or 30 μg of ethinyl oestradiol and/or at least 0.5 mg, preferably 1 mg or 2 mg of oestradiol and in each case at least 2, preferably 2, 3, 4 or 5 mg of one of gestagen components a) to k) with the exception of d), e) or f) and optionally conventional auxiliary substances.

A contraceptive action is very particularly preferably also achieved by using a hormone combination of in each case 20 μg of ethinyl oestradiol or 1 mg of oestradiol and ≧2 mg of gestagen components a) to k) with the exception of d), e) or f).

The medicament used according to the invention is preferably formulated in the form of tablets which, in addition to the stated hormone combination, optionally also contain conventional auxiliary substances. These tablets are in particular provided in the form of at least 21, preferably 21 to 25, daily units containing the hormone combination which are intended to be taken orally without interruption, followed by a 3 to 7 day interval in taking or in combination with 7 to 3 hormone-free daily units to be taken orally without interruption.

In order to alleviate or avoid menstrual cycle-dependent mood swings in addition to optional hormonal contraception, the medicament may also be provided in the form of daily units containing the hormone combination for uninterrupted administration over several years, preferably for up to 2 years, particularly preferably for up to 1 year, optionally in combination with 7 to 3 hormone-free daily units for uninterrupted administration or followed by a 7 to 3 day interval in taking.

The medicament prepared according to the invention may, however, also be provided for use according to the invention in a dosage form with fewer than 365 daily units containing the hormone combination, such as for example with 77 to 193 or 42 to 52 daily units containing the hormone combination for uninterrupted oral administration, followed by an interval in taking of 7 to 3 days or in combination with 7 to 3 hormone-free daily units for uninterrupted administration.

As already stated, the 7 to 3 hormone-free daily units may also be replaced by a correspondingly long interval in taking. The oral dosage form with the above-stated number of daily units containing the hormone combination may accordingly also assume the form of a kit which comprises two or more of these dosage forms to be taken in an ongoing manner interrupted by a corresponding interval in taking. Self-evidently, a kit may also comprise two or more oral dosage forms which [allow] daily units containing the hormone combination to be taken without interruption in combination with the stated number of hormone-free daily units to be taken without interruption.

Preferably, each of the daily units containing the hormone combination comprises the same quantity of the oestrogen component or the gestagen component, i.e. the quantity both of ethinyl oestradiol and/or oestradiol and of one of gestagen components a) to k) is kept constant over a tablet-taking cycle which, as explained above, may last for up to several years.

In a further preferred embodiment, all the daily units containing the hormone combination in each case contain ≧2 mg of one of gestagen components a) to k) with the exception of d), e) or f) and 20 μg of ethinyl oestradiol and/or 1 mg of oestradiol and are taken not only for the treatment of menstrual cycle-dependent mood swings, i.e. for alleviating or preventing them, but also as a monophasic contraceptive with daily units containing the hormone combination to be taken without interruption for 21 to 25 days followed by an interval in taking or by taking hormone-free daily units without interruption over a period of 7 to 3 days.

In a further embodiment, the daily units containing the hormone combination may in known manner vary over 21 to 25 days in terms of their content of ethinyl oestradiol or oestradiol or of gestagen components a) to k) with the exception of d), e) or f) according to a biphasic or triphasic tablet-taking cycle, if they are intended not only for the treatment of menstrual cycle-dependent mood swings but also for contraception.

To this end, the daily units containing the hormone combination preferably comprise a quantity of less than or equal to 20 μg of ethinyl oestradiol, preferably 20 μg or 30 μg of ethinyl oestradiol or 1 mg of oestradiol over all the phases and a variable quantity of 2 to 5 mg of one of gestagen components a) to k) with the exception of d), e) or f) depending on the phase.

In a biphasic contraceptive, the tablet-taking cycle preferably begins with taking a daily unit containing 2 to 3 mg of one of these gestagen components a) to k) with the exception of d), e) or f) in addition to ethinyl oestradiol or oestradiol daily over a period of 7 to 12 days, followed by taking a daily unit containing 3 to 4 mg of the same gestagen component daily over a period of 9 to 18 days, the quantity of the gestagen component always being lower in the first phase than in the second phase but in each case remaining constant, whereas the quantity of ethinyl oestradiol or oestradiol per daily unit remains constant at 20 μg or 30 μg and 1 mg respectively over both phases.

If the medicament is intended for the treatment of menstrual cycle-dependent mood swings as well as acting as a triphasic contraceptive, the tablet-taking cycle preferably begins with taking a daily unit containing 2 to 3 mg of one of gestagen components a) to k) with the exception of d), e) or f) in addition to ethinyl oestradiol or oestradiol without interruption over a period of 6 to 7 days, followed by taking a daily unit containing 3 mg of one of the stated gestagen components in addition to ethinyl oestradiol or oestradiol daily over an uninterrupted period of 5 to 9 days, and ends with taking a daily unit containing 3 to 5 mg of one of the stated gestagen components daily without interruption over a period of 5 to 14 days. In a triphasic contraceptive too, the daily units in each case contain quantities of the same gestagen component which vary from phase to phase, the particular quantity of gestagen component per daily unit rising from the first to the third phase, but remaining constant within a phase, with the quantity of ethinyl oestradiol or oestradiol likewise being kept constant over all the phases.

Both in a medicament which provides a biphasic contraceptive action and one providing a triphasic contraceptive action, the daily units containing the hormone combination preferably in each case contain 20 to 50 μg, particularly preferably in each case 30 μg, very particularly preferably in each case 20 μg of ethinyl oestradiol and/or preferably in each case 1 to 4 mg, preferably 2 mg, particularly preferably 1 mg of oestradiol. Moreover, it is of particular importance for achieving maximum reliability of contraceptive action to ensure that 21 to 25 daily units containing the hormone combination are taken in succession and without interruption per tablet-taking cycle. Once the daily units containing the hormone combination have been taken, hormone-free daily units may then immediately be taken without interruption over 7 to 3 days or a corresponding interval in taking of identical length may be observed.

Thanks to the use in particular of a monophasic medicament, it is possible according to the invention not only to alleviate but also to prevent menstrual cycle-dependent mood swings, with not only impairment of the state of mind extending as far as an emotional low point being prevented in women who suffer from menstrual cycle-dependent mood swings, but also the emotional state, i.e. the state of mind, of a woman being improved over her entire menstrual cycle in such a manner that her overall mood is uplifted.

The medicament used preferably takes the form of an oral dosage form, very particularly preferably in the form of tablets. Daily units here in each case correspond to one tablet, said daily units being packaged, preferably in blister packs, in accordance with a tablet-taking cycle, preferably with an indication of the particular daily unit to be taken, and being marketed as a medicament pack containing at least one such blister pack, preferably at least 3 blister packs for the particular number of tablet-taking cycles or for a desired uninterrupted period of administration.

### In Vitro Data

It is known that the endogenously formed neurosteroid allopregnanolone has a positive effect on mood. Plasma concentrations of allopregnanolone are reduced in female patients with a depressive mood. The positive action of allopregnanolone on mood is attributed to its interaction with GABA_{A} receptors. Allopregnanolone acts on this central nervous system receptor as a positive allosteric modulator and so results in anxiolytic and mood uplifting effects.

It has been found that 3-α-OH-CMA or 3-β-OH-CMA exhibit binding properties to GABA_{A} receptors which are very similar to those of allopregnanolone. Allopregnanolone has an influence on the binding of radioactively labelled muscimol (agonist) to these receptors.

### 1) Influence of Allopregnanolone, 3-α-OH-CMA or 3-β-OH-CMA on the Binding of Muscimol to GABA_{A} Receptors (Rat Brain)

The following determinations of the influence of allopregnanolone, 3-α-OH-CMA or 3-β-OH-CMA on the binding of muscimol to GABA_{A} receptors (rat) are based on the experimental methods published by Snodgrass, S. R. (Nature, 1979, vol. 273, p. 392-394). The corresponding disclosure is hereby introduced as a reference and is part of the disclosure of the present application.

### A) Allopregnanolone

Membrane preparations are produced from rat cerebral cortex material. The reference substance used is 5 nM of [³H] muscimol; 10 μM of muscimol are used for determining nonspecific binding. The incubation period of the substances ([³H]muscimol, allopregnanolone) on the receptor amounts in each case to 10 minutes at a temperature of approx. 4° C. The stock solution (5×10⁻² M) of allopregnanolone is in each case prediluted 1:10 in 75% strength DMSO and then further diluted 1:5 in 25% strength DMSO. The final concentration of allopregnanolone in the test is in each case 1×10⁻¹⁰ M, 1×10⁻⁹ M, 1×10⁻⁸ M, 1×10⁻⁷ M, 1×10⁻⁶ M and 1×10⁻⁵ M. After the above-stated incubation period of the substances, the incubation batches are filtered using standard methods and washed and the radioactivity of the filters is determined with a scintillation counter. Such standard methods are known to a person skilled in the art. The experiments are in each case carried out on duplicate batches.

It can be shown that, at a concentration of 1 μM of allopregnanolone, binding of the radioactively labelled muscimol (5 nM) is increased by 38%.

### B) 3-α-OH-CMA or 3-β-OH-CMA

The influence of 3-α-OH-CMA or 3-β-OH-CMA on the binding of muscimol to GABA_{A} receptors (rat) is determined in a manner similar to the above-described method. 3-α-OH-CMA or 3-β-OH-CMA is used instead of allopregnanolone.

It can be shown that, at a concentration of 0.1 μM of 3-β-OH-CMA, binding of the radioactively labelled muscimol (5 nM) is increased by 31% and, at a concentration of 0.001 μM of 3-α-OH-CMA, it is increased by 12%.

It may be concluded on this basis that 3-α-OH-CMA or 3-β-OH-CMA have a mood uplifting effect.

2) Determination of the Affinity of 3-α-OH-CMA or 3-β-OH-CMA for the human Progesterone Receptor

The following determination of the affinity of 3-α-OH-CMA or 3-β-OH-CMA for the human progesterone receptor is based on the experimental methods published by Eckert et al. (Cancer Research, 1982, vol. 42, pp. 139-144). The corresponding disclosure is hereby introduced as a reference and is part of the disclosure of the present application.

The cytosolic fractions of MCF-7 cells, which contain the human progesterone receptor, are used. The reference substance used is 2 nM of [³H] R 5020; 1 μM of R 5020 is used for determining nonspecific binding. The incubation period of the substances ([³H] R 5020, 3-α-OH-CMA or 3-β-OH-CMA) on the receptor amounts in each case to 20 hours at a temperature of approx. 4° C. Stock solutions (5×10⁻² M) of 3-α-OH-CMA or 3-β-OH-CMA are in each case prediluted 1:10 in 75% strength DMSO and then further diluted 1:5 in 25% strength DMSO. The final concentrations of 3-α-OH-CMA or 3-β-OH-CMA in the test are 3×10⁻¹⁰ M, 3×10⁻⁹ M, 1×10⁻⁸ M, 3×10⁻⁸ M, 1×10⁻⁷ M, 3×10⁻⁷ M, 1×10⁻⁶ M and 1×10⁻⁵ M. After the above-stated incubation period of the substances, the incubation batches are filtered using standard methods and washed and the radioactivity of the filters is determined with a scintillation counter. Such standard methods are known to a person skilled in the art. The experiments are in each case carried out on duplicate batches.

The corresponding IC₅₀ values are calculated by nonlinear regression analysis of the displacement curves using the Hill curve fitting formula. A person skilled in the art is aware of such a calculation procedure.

The corresponding Kᵢ values (inhibition constants) are determined using the Cheng-Prusoff equation (Kᵢ=IC₅₀/(1+(L/K_{D})), where L denotes the concentration of the radioligand in the test and K_{D} the affinity of the radioligand for the receptor).

The measured values are set out in the Table below.

### 3) Determination of the Affinity of 3-α-OH-CMA or 3-β-OH-CMA for the Human Androgen Receptor

The following determination of the affinity of 3-α-OH-CMA or 3-β-OH-CMA for the human androgen receptor is based on the experimental methods published by Zava et al. (Endocrinology, 1979, vol. 104, pp. 1007-1012). The corresponding disclosure is hereby introduced as a reference and is part of the disclosure of the present application.

The cytosolic fractions of LNCaP cells, which contain the human androgen receptor, are used. The reference substance used is 0.5 nM of [³H]methyltrienolone; 1 μM of mibolerone is used for determining nonspecific binding. The incubation period of the substances ([³H]methyltrienolone, 3-α-OH-CMA or 3-β-OH-CMA) on the receptor amounts in each case to 24 hours at a temperature of approx. 4° C. The stock solutions, the dilution series of 3-α-OH-CMA or 3-β-OH-CMA, the washing steps, the determination of radioactivity, and the methods for calculating the particular IC₅₀ and Kᵢ values match the experimental protocol described in point 2).

The measured values are set out in the Table below.

| | | |
|---|---|---|
| Substance | IC₅₀ [nM] | Kᵢ [nM] |

Progesterone receptor (human)

| | | |
|---|---|---|
| 3-α-OH-CMA | 39 | 13 |
| 3-β-OH-CMA | 18 | 6 |

Androgen receptor (human)

| | | |
|---|---|---|
| 3-α-OH-CMA | 100 | 83 |
| 3-β-OH-CMA | 25 | 20 |

The data reveal that both 3-α-OH-CMA and 3-β-OH-CMA have an elevated affinity for the human progesterone receptor and for the human androgen receptor, their particular contraceptive and antiandrogenic action being derivable therefrom.

### EXAMPLES

### a) Production of the Medicament

### Example 1

### Composition

| | |
|---|---|
| Per tablet | Per batch |

| | | | | |
|---|---|---|---|---|
| Ethinyl oestradiol | 0.020 | mg | 0.0020 | kg |
| 3α-Hydroxychlormadinone acetate | 3.000 | mg | 0.3000 | kg |
| 3β-Hydroxychlormadinone acetate | 2.000 | mg | 0.2000 | kg |
| Povidone K30 | 3.000 | mg | 0.3000 | kg |
| Lactose | 31.980 | mg | 3.1980 | kg |
| Maize starch | 0.500 | mg | 1.0500 | kg |
| Highly disperse silicon dioxide | 0.500 | mg | 0.0500 | kg |

Ethinyl oestradiol (EO) and Povidone K 30 (PVP) were dissolved in 600 ml of ethanol. 3α-Hydroxychlormadinone acetate and 3β-hydroxychlormadinone acetate (particle size 90%<50 μm), lactose and maize starch were mixed in a mixer/pelletiser (Diosna P25) for 5 minutes and then moistened thoroughly and mixed with the ethanolic EO/PVP solution. The moist composition was forced through a 3 mm screen and dried in a vacuum drying cabinet. The dried granular product was disagglomerated through a 0.6 mm screen, mixed with highly disperse silicon dioxide and pressed on a tablet press with 5 mm punches into tablets with a weight of 50 mg.

The tablets were coated with a methylhydroxypropylcellulose-based coating of the following composition (coating mass 2 mg per tablet)

| | | |
|---|---|---|
| Methylhydroxypropylcellulose 6 mPa · s | 0.1351 | kg |
| Polyethylene glycol 6000 | 0.0395 | kg |
| Propylene glycol | 0.0054 | kg |
| Purified water | 1.6200 | kg |

In each case 24 coated tablets as the daily units containing the hormone combination and in each case 4 correspondingly composed, hormone-free, coated tablets were packaged in a blister pack.

### Example 2

Tablets of the following composition were produced in a manner similar to Example 1

| | |
|---|---|
| Per tablet | Per batch |

| | | | | |
|---|---|---|---|---|
| Ethinyl oestradiol | 0.030 | mg | 0.0030 | kg |
| Chlormadinone acetate | 1.000 | mg | 0.1000 | kg |
| 3α-Hydroxychlormadinone | 2 | mg | 0.2000 | kg |
| acetate | | | | |
| 3β-Hydroxychlormadinone | 1 | mg | 0.1000 | kg |
| acetate | | | | |
| 3α-Hydroxy-17β-acetoxy-5β- | 1 | mg | 0.1000 | kg |
| pregnan-20-one | | | | |
| Povidone K30 | 3.000 | mg | 0.3000 | kg |
| Lactose | 31.970 | mg | 3.1970 | kg |
| Maize starch | 0.500 | mg | 0.0500 | kg |
| Highly disperse silicon dioxide | 0.500 | mg | 0.0500 | kg |

As stated in Example 1, the tablets were provided with the coating described in Example 1 (coating mass 2 mg per tablet).

In each case 24 coated tablets as the daily units containing the hormone combination and in each case 4 correspondingly composed, hormone-free, coated tablets were packaged in a blister pack.

1. A method for alleviating menstrual cycle-dependent mood swings and optionally hormonal contraception in women comprising administering to a woman a medicament comprising a hormone combination of at least one oestrogen selected from the group consisting of ethinyl oestradiol (I) and oestradiol (II) as oestrogen component and at least one metabolite of chlormadinone acetate selected from the group consisting of 3α-hydroxy-6-chloro-17α-acetoxy-4,6-pregnadien-20-one (3α-hydroxychlormadinone acetate), 3β-hydroxy-6-chloro-17α-acetoxy-4,6-pregnadien-20-one (3β-hydroxychlormadinone acetate), 3α-hydroxy-17α-acetoxy-5β-pregnan-20-one and 3β-hydroxy-17α-acetoxy-5β-pregnan-20-one, optionally mixed with chlormadinone acetate as gestagen component.

2. The method according to claim 1, wherein the medicament is provided in the form of daily units.

3. The method according to claim 1, wherein a daily unit is produced by using a hormone combination comprising of 5 to 50 μg of the oestrogen component (I) and/or 0.5 to 4 mg of the oestrogen component (II) and 1 to 10 mg of the gestagen component and optionally conventional auxiliary substances.

4. The method according to claim 4, wherein a daily unit is produced by using a hormone combination comprising of 5 to 30 μg of the oestrogen component (I) and/or 0.5 to 2 mg of the oestrogen component (II) and 1 to 10 mg of the gestagen component and optionally conventional auxiliary substances.

5. The method according to claim 1, wherein gestagen component is selected from the following,
a) 3α-hydroxychlormadinone acetate or
b) 3β-hydroxychlormadinone acetate or
c) a mixture of a) and b) in any desired mixing ratio or
d) 3α-hydroxy-17α-acetoxy-5β-pregnan-20-one or
e) 3β-hydroxy-17α-acetoxy-5β-pregnan-20-one or
f) a mixture of d) and e) in any desired mixing ratio or
g) a mixture of a) and/or b) with d) and/or e) in any desired mixing ratio or
h) a mixture of chlormadinone acetate with a) and/or b) in a mixing ratio of 10 to 90 wt. % of chlormadinone acetate and 90 to 10 wt. % of a) and/or b), relative to the total mixture, or
i) a mixture of chlormadinone acetate with d) and/or e) in a mixing ratio of 10 to 90 wt. % of chlormadinone acetate and 90 to 10 wt. % of d) and/or e), relative to the total mixture, or
j) a mixture of chlormadinone acetate with c) and f) in a mixing ratio of 10 to 90 wt. % of chlormadinone acetate and 90 to 10 wt. % of c) and f), relative to the total mixture, or
k) a mixture of chlormadinone acetate with g) in a mixing ratio of 10 to 90 wt. % of chlormadinone acetate and 90 to 10 wt. % of g), relative to the total mixture.

6. The method according to claim 5, comprising daily units of a hormone combination wherein the daily units each comprise at least 15 μg, ethinyl oestradiol and/or at least 0.5 mg, and at least 1, one of gestagen components a) to k) and optionally conventional auxiliary substances.

7. The method according to claim 6, wherein the hormone combination in a daily unit comprises of 20 μg of ethinyl oestradiol and/or 1 mg of oestradiol and ≧2 mg of the gestagen component a) to k) with the exception of d), e) or f).

8. The method according to claim 1, wherein the medicament is provided in the form of at least 21 daily units containing the hormone combination for uninterrupted administration optionally followed by a 7 to 3 day interval in taking or by 7 to 3 hormone-free daily units for uninterrupted administration.

9. The method according to claim 8, wherein the medicament is provided in the form of daily units containing the hormone combination for uninterrupted administration over several years, 1 year, and of 7 to 3 hormone-free daily units for uninterrupted administration or followed by an interval in taking of 3 to 7 days.

10. The method according to claim 8, wherein the medicament is provided in the form of 77 to 193 daily units containing the hormone combination for uninterrupted administration and of 7 to 3 hormone-free daily units for uninterrupted administration or followed by an interval in taking of 3 to 7 days.

11. The method according to claim 8, wherein the medicament is provided in the form of 42 to 52 daily units containing the hormone combination for uninterrupted administration and of 7 to 3 hormone-free daily units for uninterrupted administration or followed by an interval in taking of 3 to 7 days.

12. The method according to claim 8, wherein the medicament is provided in the form of 21 to 25 daily units containing the hormone combination for uninterrupted administration and of 7 to 3 hormone-free daily units for uninterrupted administration or followed by a 3 to 7 day interval in taking.

13. The method according to claim 5, wherein, in each of the daily units containing the hormone combination, the medicament comprises quantitatively the same combination of ethinyl oestradiol or oestradiol and a gestagen component a) to k).

14. The method according to claim 1 wherein the medicament prevents an impairment of the state of mind.

15. The method according to claim 1 wherein the medicament offsets menstrual cycle-dependent mood swings.

16. The method according to claim 1 wherein the medicament improves the state of mind and uplifts mood over an entire female menstrual cycle.

17. (canceled)

18. The method according to claim 7 wherein the hormone combination in a daily unit comprises 30 μg of ethinyl oestradiol.

19. The method according to claim 9 wherein the medicament is provided in the form of daily units containing the hormone combination for uninterrupted administration for up to 2 years.

20. The method according to claim 19 wherein the medicament is provided in the form of daily units containing the hormone combination for uninterrupted administration for up to 1 year.
